(19) United States
(12) Patent Application Publication
(10) Pub. No.: US 2004/0199174 A1
(43) Pub. Date: Oct. 7, 2004

(54) Device for folding an intraocular lens, and storage system for an intraocular lens

(76) Inventors: Thomas Herberger, Forchheim (DE); Gerd Van Der Heyd, Adelsdorf (DE); Arthur Messner, Schnaittach (DE)

Correspondence Address: LERNER AND GREENBERG, P.A., POST OFFICE BOX 2480, HOLLYWOOD, FL, 33022-2480, US

(21) Appl. No.: 10/823,250

(22) Filed: Apr. 12, 2004

Related U.S. Application Data
(63) Continuation of application No. PCT/EP02/11434, filed on Oct. 11, 2002.

(30) Foreign Application Priority Data
Oct. 12, 2001 (DE) 201 16 676.3
Dec. 29, 2001 (DE) 101 64 420.5

(51) Int. Cl. A61F 2/16; A61F 9/007

(52) U.S. Cl. 606/107; 623/6.12

(57) ABSTRACT

A device is part of an injection system for injecting a foldable intraocular lens (IOL) into a human eye. The device contains a folding area with two pivotably interconnected half-shells which can be pivoted from an open starting position to a closed finishing position. A sheet-like band loop is provided which is secured with a securing end on the first half-shell and is guided displaceably on the second half-shell. In the open starting position, a receiving space for the IOL is formed between the band loop and the two half-shells. By pulling on the displaceably guided band loop, the half-shells can be moved in the direction toward their finishing position. The band loop on the one hand permits reliable and gentle folding of the IOL. At the same time, the band loop permits storage of the IOL, in the unfolded state, in the folding area.

### CROSS-REFERENCE TO RELATED APPLICATION

This application is a continuation, under 35 U.S.C. § 120, of copending international application No. PCT/EP02/11434, filed Oct. 11, 2002, which designated the United States; this application also claims the priority, under 35 U.S.C. § 119, of German patent application No. 201 16 676.3, filed Oct. 12, 2001, and German patent application No. 101 64 420.5, filed Dec. 29, 2001; the prior applications are herewith incorporated by reference in their entirety.

### BACKGROUND OF THE INVENTION

Field of the Invention

The invention relates to a device for folding an intraocular lens, having a folding area with two pivotably interconnected half-shells which can be pivoted from an open starting position to a closed finishing position in which they enclose between them a guide channel for the intraocular lens. The invention relates further to a storage system for an intraocular lens.

The device is used for implanting a foldable intraocular lens (hereinafter referred to as IOL). The device, optionally combined with further components to form an implantation system, is intended to permit implantation of a temporarily folded IOL through an incision opening of approximately 3 mm into the capsular bag of a patient suffering from gray cataract.

In operations of the above type, the size of the incision needed for introducing the foldable IOL into the eye is of great importance. To ensure an optimal healing process, this incision should be as small as possible (e.g. 3 mm). It is only through the possibility of “folding” the IOL that one is able to meet this requirement. In general, there are three different systems or methods for implanting the IOL.

In a first system, a container is used in which the IOL is folded, then removed with forceps and then taken up in the folded state by the implantation forceps and introduced into the capsular bag. German Patent DE 40 39 119 C1, corresponding to U.S. Pat. No. 5,139,501, describes a container for this system, which container is suitably configured for folding the IOL. In this system, however, the risk of dropping the lens must be considered since the latter, in the folded state, is under mechanical tensioning. In addition, the operating method requires practice.

According to a second system, the IOL is folded in a folding device, and the folding device is inserted, together with the folded IOL, into an injection aid, a so-called injector. The injector provides for the axial forward drive and, if appropriate, a further cross-sectional reduction of the lens to be injected. Such a system is described, for example, in European Patent EP 0 785 760 B1, corresponding to U.S. Pat. No. 5,810,833, or in U.S. Pat. No. 4,681,102. According to International Patent Disclosure WO 96/15743, the insertable folding device is also to be used as the container for the lens so that it is not necessary to move the IOL from a separate container into the folding device, and the risk of dropping it, which is otherwise present, does not arise. However, during the folding procedures taking place in the folding devices, it is not possible to exclude the possibility of damage to the implant, and this damage can only be established when the implant is sited in the eye of the patient.

In a third injection system, the folding device and injector form a unit, so that the fitting procedure is dispensed with. Such a system is described for example in Published, Non-Prosecuted German Patent Application DE 36 10 925 A1. Maneuvering systems for an IOL, in which the latter is held in a defined shape or brought into this defined shape with the aid of a thin film or band, are known, for example, from U.S. Pat. Nos. 5,976,150 and 4,917,680.

### SUMMARY OF THE INVENTION

It is accordingly an object of the invention to provide a device for folding an intraocular lens, and a storage system for an intraocular lens which overcomes the above-mentioned disadvantages of the prior art devices of this general type, which ensures reliable and gentle folding of the IOL and a high level of safety of the operation.

With the foregoing and other objects in view there is provided, in accordance with the invention, a device for folding an intraocular lens. The device contains a folding area having two pivotably interconnected half-shells which can be pivoted from an open starting position to a closed finishing position in which the half-shells enclose between them a guide channel for receiving the intraocular lens. The half-shells include a first half-shell and a second half-shell. A sheet-like band loop has a securing end secured on the first half-shell and is guided displaceably by the second half-shell, and in the open starting position, a receiving space for the intraocular lens is formed between the band loop and the two half-shells such that, with the intraocular lens fitted in place, the intraocular lens is surrounded by the band loop and the two half-shells in the open starting position. And in that, by pulling on the band loop, the two half-shells are moved in a direction toward the closed finishing position such that the intraocular lens is folded.

Here, the device has, in a folding area, two pivotably interconnected half-shells which can be pivoted from an open starting position to a closed finishing position. In the closed finishing position, the two half-shells enclose between them a guide channel for the IOL. Furthermore, the device is provided with a sheet-like band loop which is secured with a securing end on the first half-shell and is guided displaceably on the second half-shell. In the open starting position, a receiving space for the IOL is formed between the band loop and the two half-shells. By pulling on the displaceably guided band loop, the half-shells can be moved in the direction toward their finishing position, that is to say toward one another. Thus, the band loop permits or assists the folding of the IOL in a very simple way.

With a device of this kind, particularly reliable and gentle folding of an IOL is ensured. The latter is first fitted into the receiving area and already in this state it is safely held and protected by the band loop that closes off the receiving space at the top. In the next stage, the band loop is pulled in a pull direction so that the two half-shells are pivoted toward one another. The IOL placed in the receiving space is thus automatically folded. Since, in the starting position, the IOL is already clamped to some extent between the half-shells and the band loop, the IOL cannot slip.

In a preferred development, the two half-shells in the closed finishing position bear against one another via their outer longitudinal edges. At the same time, the band loop is secured with its securing end on the longitudinal edge of the first half-shell and is guided on the longitudinal edge of the second half-shell. This has the advantage that, as the two half-shells are folded together, the receiving space delimited at the top by the band loop steadily and continuously reduces to the guide channel formed in the finishing position. It is therefore possible to rule out a situation in which, when the two half-shells are folded together, a subsidiary portion of the IOL is clamped between the two longitudinal edges.

For reliable guiding of the band loop, a guide slit is preferably provided in the area of the second half-shell.

According to an expedient development, and viewed in a cross section perpendicular to the longitudinal direction of the half-shells, the inner side of one of the two half-shells is provided with a step. This acts on the one hand in the manner of a limit stop on which the still unfolded IOL bears and is held fixed in position before the actual folding procedure. The step at the same time represents an abrupt reduction in the cross-sectional diameter. In the folding process, one end of the IOL bears on the step, and the IOL is as it were rolled up until its second end likewise reaches the step area. The second end can advantageously be guided past the radially further outwardly lying first end of the IOL, so that the IOL as a whole is wound up like a spiral.

To form the step which permits support of an edge of the IOL, a side flap is expediently provided which extends along the channel axis and which, from a forward or outer position, can be guided radially, that is to say toward the center of the receiving space, into a retracted position. By being displaceable or pivotable, the side flap can be pushed in the direction toward the channel axis of the folding area after successful folding, so that the step is reversed and continuous and smooth surfaces are present both in the inside of the channel axis and also on the outer circumference of the folding area. For a reliable folding process, the side flap can be locked in the retracted position.

According to an advantageous alternative embodiment, the securing end is adjoined by a subsidiary portion of the band loop which, at a kink oriented parallel to the guide channel, is connected in an articulated manner to the remaining portion of the band loop, and which, in the open starting position, is oriented outward in the radial direction, that is to say pointing away from the half-shell. In this way, a pocket extending above the first half-shell is formed. One end of the initially unfolded IOL is placed into this pocket. In the folding procedure, that is to say when pulling on the band loop, the securing end on the one hand and the transition point between subsidiary portion and remaining portion, on the other hand, in each case form a kink having the function of a pivot axis. The end of the IOL placed in the pocket is in this way particularly reliably brought into a predefined curvature.

According to the invention, the object is moreover achieved by a storage and transport system for an IOL in which a container is provided for receiving and storing the device according to the invention. The IOL is placed, in the open starting position, into the receiving space of the folding area.

By the configuration of the band loop, the folding area can also be used particularly simply and reliably as a storage site for the IOL, since the IOL can be slightly clamped even in the open starting position between the two half-shells and the band loop, so that it is held secure. In the case of hydrophilic IOLs, the container is expediently filled with a suitable storage liquid into which the device and the IOL are placed.

According to a preferred development, the folding area is configured in one piece with an injection channel and, in a further preferred alternative embodiment, it is configured in one piece with an injector housing of an injector. The term “one piece” is to be understood here as meaning that the individual elements form a nondetachable structural unit. Because of the special configuration of the folding area with the band loop, each of these structural units is also suitable for the described storage and transport purpose. With a view to hygiene requirements, the structural units are configured in particular as disposable articles.

Other features which are considered as characteristic for the invention are set forth in the appended claims.

Although the invention is illustrated and described herein as embodied in a device for folding an intraocular lens, and a storage system for an intraocular lens, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1A is a diagrammatic, perspective view of a one-piece structural unit containing a folding area and of an adjoining injection channel, in a first variant, according to the invention;

FIG. 1B is a perspective view of a structural unit according to FIG. 1A, in a second variant;

FIG. 2 is perspective view of an injector whose injector housing, configured as a one-piece structural unit, contains a push channel, the folding area and the injection channel;

FIGS. 3 to 9 are cross-sectional views through a folding area of the variant according to FIG. 1A, in order to illustrate a procedure for folding the IOL;

FIGS. 10 to 15 are cross-sectional views through a folding area of the further variant according to FIG. 1B, in order to illustrate the procedure for folding the IOL;

FIGS. 16 to 19 are cross-sectional views showing different embodiments of an injection channel;

FIG. 20 is a perspective view of an injector housing which is provided for insertion of a structural unit according to FIG. 1A or FIG. 1B;

FIG. 21 is a perspective view of a forceps geometry for inserting the IOL into the folding area;

FIG. 22 is a perspective view of a container for storing an IOL inserted in a folding area;

FIGS. 23 to 26 are sectional views showing different embodiments of a plunger;

FIG. 27 is a perspective view of a front end of an injection channel; and

FIGS. 28 to 30 are sectional views showing different embodiments of the front end of the injection channel.

### DESCRIPTION OF THE PREFERRED EMBODIMENTS

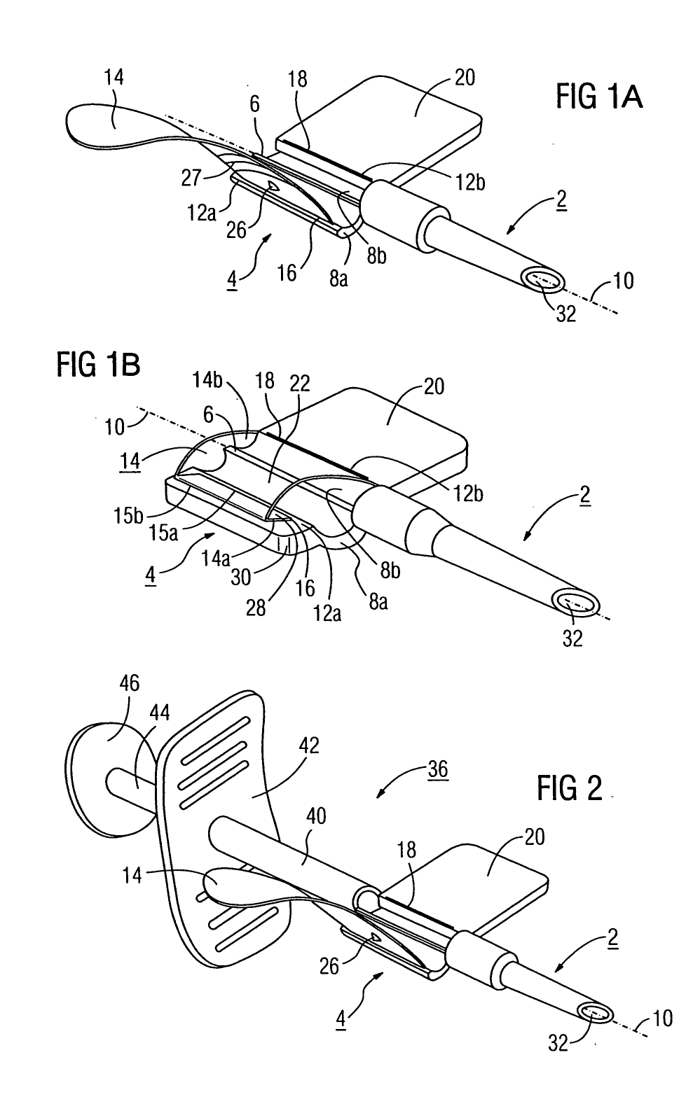

In all the figures of the drawing, sub-features and integral parts that correspond to one another bear the same reference symbol in each case. Referring now to the figures of the drawing in detail and first, particularly, to FIGS. 1A and 1B thereof, there are shown two one-part structural units that contain an injection channel 2 and a folding area 4 which are fixedly connected to one another and are a one-piece injection-molded plastic component. The folding area 4 has two half-shells 8a, 8b that are connected to one another via a hinge 6 and extend along a channel axis 10. The half-shells 8a, 8b are connected via the hinge 6 at their mutually facing longitudinal edges. At their outer longitudinal edges 12a, 12b directed away from one another, a sheet-like band loop 14 is secured with its securing end 16 on one half-shell 8a (hereinafter called the first half-shell 8a). On the other half-shell 8b (hereinafter called the second half-shell 8b), a guide slit 18 for the band loop 14 is incorporated in the area of the longitudinal edge 12b. The guide slit 18 being enclosed between the second half-shell 8b and a grip piece 20 adjacent to the latter. The band loop 14 is a sheet-like and flexibly elastic planar element, preferably of plastic.

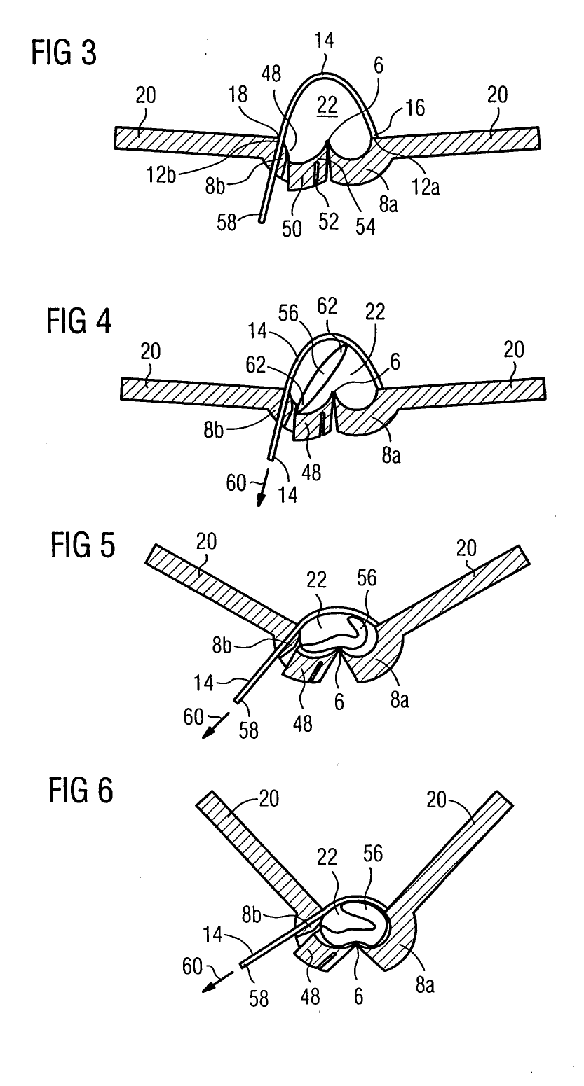

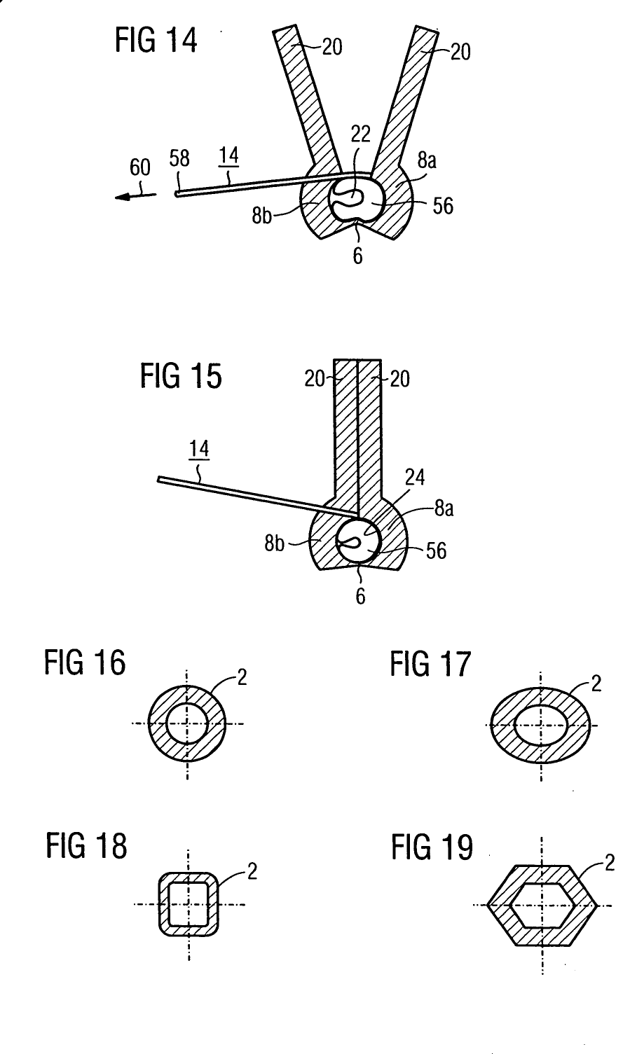

The band loop 14 is guided through the guide slit 18 so that a receiving space 22 for an intraocular lens 56 (IOL, see FIGS. 3 to 16) is formed between the half-shells 8a, 8b. By pulling on the band loop 14, the first half-shell 8a is pivoted about the pivot axis defined by the hinge 6 toward the second half-shell 8b until the two outer longitudinal edges 12a, 12b lie on one another. The two half-shells 8a, 8b are thus converted from their open starting position to a closed finishing position in which they enclose between them a guide channel 24. The receiving space 22 thus merges into the guide channel 24 (for closed finishing position, see FIGS. 8, 9 and 15).

In the area of the securing end 16, the band loop 14 has a catch recess 26 (FIG. 1A) with which it can cooperate on the outside of the second half-shell 8b or of the grip piece 20 with a corresponding non-illustrated catch element, so that the band loop 14 can be secured in a predetermined position. Transversely extending locking ridges 27 are likewise disposed on the outside of the band loop 14 and interact with the guide slit 18 in such a way that the band loop 14 can no longer slip back through the guide slit 18 counter to the pull direction.

In addition, the band loop 14 can also have further structures assisting the folding procedure, for example a roughened surface on its inside facing toward the receiving space 22.

In the first embodiment variant according to FIG. 1A, the band loop 14 is configured to be free of kinks and edges and extends approximately in a tangential direction relative to the semicircularly shaped first half-shell 8a. By contrast, in the embodiment variant according to FIG. 1B, the band loop 14 is divided into a subsidiary portion 14a and a remaining portion 14b. By this configuration, two kinks 15a, 15b oriented parallel to the guide channel 24 are formed, each of which act in the manner of a hinge. In the open starting position shown, in which the two half-shells 8a, 8b lie folded open next to one another, the subsidiary portion 14a extends outward in an approximately radial direction from the hinge 6, so that a pocket 28 is formed between the subsidiary portion 14a and the remaining portion 14b, which pocket overlaps the first half-shell 8a on the outside. In the illustrative embodiment according to FIG. 1B, the first half-shell 8a is adjoined by a grip stub 30.

The folding area 4, which can also be referred to as the loading area, is adjoined in the direction of the channel axis 10 by the injection channel 2, which has an injection tip 32 at the distal end, that is to say at the front end remote from the folding area 4. The injection channel 2 has a narrowing of its external diameter in the direction toward the injection tip 32, which narrowing is stepped (FIG. 1A) or conical (FIG. 1B). An internal diameter of the injection channel 2 can likewise narrow, preferably in a continuous and smooth manner, which is to say without edges and steps.

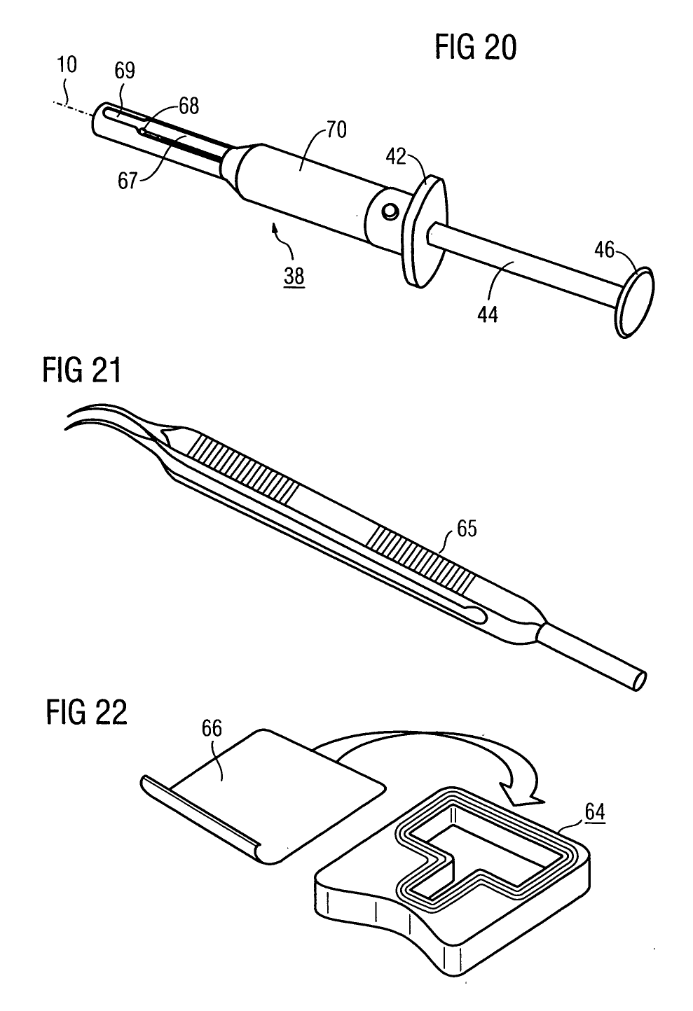

The structural unit formed by the folding area 4 and by the injection channel 2 serves for insertion into an injector housing 38, as is shown in FIG. 20 for example.

In a preferred embodiment according to FIG. 2, an injector 36 is provided in which the injector housing 38 formed of a push channel 40 and an abutment 42 is configured, together with the folding area 4 and the injection channel 2, as a one-piece structural unit and in particular as an injection-molded component. A push rod 44 that has a pressure plate 46 is guided in the push channel 40.

FIGS. 3 to 9 show a cross section through the folding area 4 similar to the embodiment shown in FIG. 1A. Here, both half-shells 8a, 8b are adjoined by grip pieces 20 which are used to make handling easier. The second half-shell 8b has, on its inner surface, a radial step 48 that serves as a kind of limit stop for the IOL 56 and assists the procedure of folding the latter. In the illustrative embodiment, the radial step 48 is formed by a side flap 50 that is pivotable in the radial direction into the receiving space 22. For this purpose, the side flap 50 is connected by another hinge 54 to the second half-shell 8b only on one side, so as to leave a compensation space 52. After closure of the half-shells 8a, 8b, the side flap 50 makes it possible to remove the step 48 again by pivoting the side flap 50 inward, so that there is no point of discontinuity either on the inside or on the outside.

The procedure for folding the IOL 56 in the embodiment variant according to FIG. 1A is explained below with reference to FIGS. 3 to 9. While FIG. 3 shows the folding area 4 still in the starting state without the IOL 56, in FIG. 4 the still unfolded IOL 56 has been placed in the receiving space 22 and bears with its one lens end 62 on the step 48. It is placed in this position by the clinical staff prior to the implantation. However, it is preferably stored in this position so that insertion of the IOL 56 into the folding area 4 by the clinical staff during the operation is unnecessary, thus avoiding errors in the handling of the IOL 56. In the next stage, a suitable amount of viscoelastic material is introduced into the receiving space 22 surrounding the IOL 56. This can be done both through additional filler bores and through the other openings already present. By this measure, it is possible to improve the subsequently required sliding property of the folded IOL 56 in the injection channel 2 and on its way there.

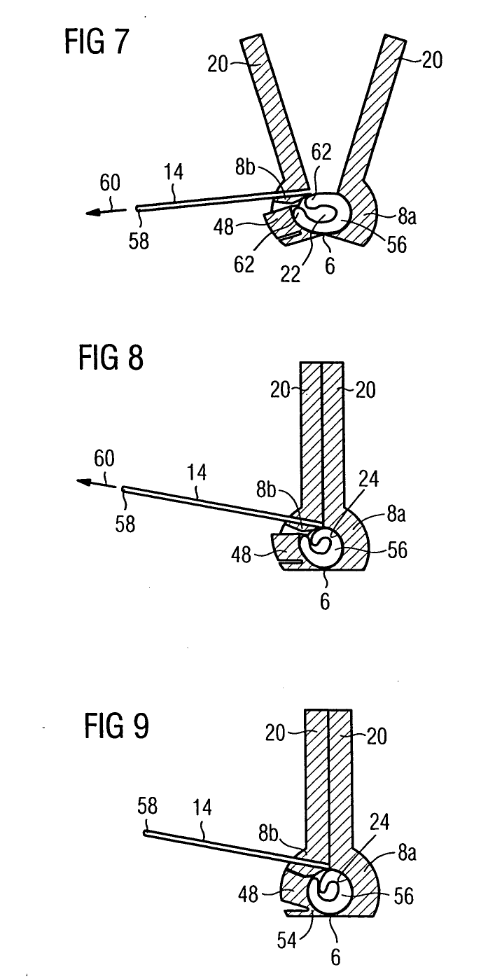

In the next stage, a free end 58 of the band loop 14 is pulled in pull direction 60. The diagrams in FIGS. 5 to 9 show how the actual folding of the IOL 56 proceeds. First, the IOL 56 buckles in the direction of the first half-shell 8a shown on the right in FIG. 5. From the subsequent figures, it will be seen how the cross-sectional area offered to the IOL 56 in the folding area 4 is still further reduced by continued pulling. FIGS. 7 and 8 show how the lens ends 62 slide past one another, because of the step 48 present in the second half-shell 8b, and come to lie roughly in a spiral shape as shown in FIG. 8. By actuation from outside, the side flap 50 can now be pressed so far in the direction of the channel axis 10 until a continuous channel profile is obtained. The folding area 4 is now in its closed finishing position, in which the guide channel 24 is formed by the half-shells 8a, 8b bearing on one another. The band loop 14 can then be secured on the outside of the second half-shell 8b via the catch recess 26 shown in FIG. 1A.

The exact way in which the IOL 56 gets from the unfolded state to the folded state is dependent on a large number of factors such as, for example, the lens geometry. However, it is always the case that the IOL 56 lies under the band loop 14 throughout the entire folding procedure, which eliminates the risk of lens parts becoming stuck. The way in which the folding specifically proceeds has no effect on this property, which fact thus also permits folding of a very wide variety of IOL types.

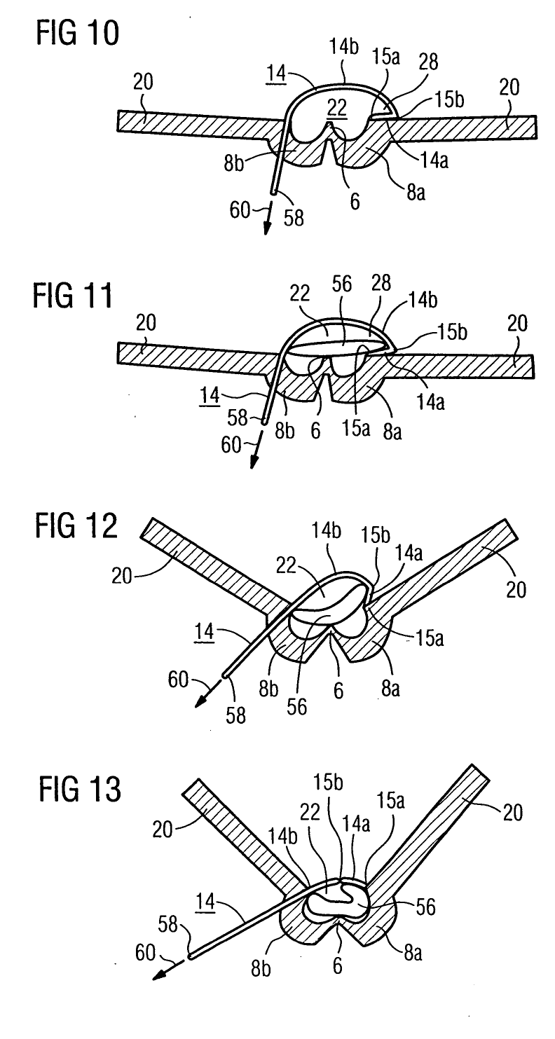

The folding procedure in the alternative embodiment variant according to FIG. 1B is explained with reference to FIGS. 10 to 15. The side flap 50 shown in FIGS. 3 to 9 is not necessary in this embodiment variant. The alternative embodiment variant additionally has the advantage that the band loop 14 from the outset extends with its subsidiary portion 14a horizontally in the radial direction away from the receiving space 22 and has the two kinks 15a, 15b. The IOL 56 is initially in the starting position shown in FIG. 11. In this starting position, the IOL 56 can already be under slight mechanical prestressing, which facilitates the rolling in the desired direction. By continued pulling on the end 58 of the band loop 14, a rolling movement of the IOL 56 in a defined direction is initiated via a pocket 28 of the band loop 14 defined by the kinks 15a, 15b (see FIG. 12). FIGS. 13 to 15 show how the folding procedure continues in this alternative embodiment.

Since the folding method described here with the band loop 14 permits a reduction in cross section of the IOL 56 to the minimum dimension achievable, it is not strictly necessary, in contrast to other injection systems, to have a conical continuation of the injection channel 2 for further folding of the IOL 56. Thus, the danger of destroying the IOL 56 during the final axial forward movement in the injection channel 2 is reduced to a minimum. However, the use of a conical injection channel 2 is still in principle possible and also independent of the cross-sectional shape thereof.

In addition to the particularly reliable and gentle folding of the IOL 56, the use of the band loop 14 has the additional advantage that the IOL 56 is held securely in the receiving space 22 even in the open starting position, as is shown in FIG. 4 or 11. Thus, the folding area 2 with the band loop 14 is also particularly advantageously suitable for storage and transport of the IOL 56. To ensure that the IOL is held sufficiently securely in the receiving space 22, the band loop 14 can be tightened slightly and locked in this state via the locking ridges 27, so that the IOL 56 is slightly prestressed and thus clamped in the receiving space 22.

This storage in the folding area 4 is possible both with hydrophobic and hydrophilic foldable materials. In the latter variant, a suitable container system is provided which rules out dehydration of the IOL 56 during storage. A configuration of a container 64 in such a container system is shown in FIG. 22. In the container 64 shown, escape of the liquid is prevented by sealing with a metal foil 66 secured by welding, for example ultrasound welding, but preferably thermal welding. The container 64 at the same time also serves as a packaging for transport. Moreover, the inner contour of the container 64 is configured in such a way that the folding area 4 containing the IOL 56 has little possibility of movement. The storage and transport of the IOL 56 are also possible in the configuration shown in FIG. 2. In this embodiment, the folding area 4 and the injection channel 2 (FIG. 1A or 1B) are integrated. All of these methods are done without manual individual handling of the IOL 56 for fitting it into the folding area 4. The last-mentioned variant with storage in the injector 36 is particularly user-friendly and, in conjunction with a disposable injector, is also advantageous from the point of view of sterility, because the clinical staff only have to remove the injector 36 with the fitted IOL 56 from a sterile protective package and then fold the IOL 56 by pulling on the band loop 14.

Of course, the IOL 56 can alternately be stored in a conventional manner in a separate container from which it then has to be removed by the clinical staff and fitted into the folding area 4. In this case too, it is not important from which foldable material the IOL 56 is made. The fitting is done with the aid of a suitable maneuvering instrument, in particular forceps 65, as shown in FIG. 21.

As regards the geometry of the individual elements, it is possible to use different geometry variations. In this connection, it is necessary to minimize not only the risk of damage to the IOL 56 during handling, but also the risk of damage to the eye of the patient. For this purpose, the end of the injection channel 2 directed toward the patient has a smooth geometry. In the case of the step 48 used for folding according to FIG. 3, this therefore does not continue as far as the distal end, i.e. to the injection tip 32. For this purpose, as has been explained with reference to FIGS. 3 to 9, the movable side flap 50 is provided. The advantage afforded by this is that the geometry of a plunger 68 (see FIG. 20) can be effectively adapted to the inner geometry of the injection channel 2 shown in FIG. 9. The reversal of the step 48 takes place by inserting the device in the injector, the inner contour of the injector being configured so that the movable side flap 50 is pressed radially in the direction of the channel axis 10 and the step 48 disappears. If an integrated embodiment is used, as shown in FIG. 2, the step 48 is reversed with the aid of a dish-shaped element that is pushed over the injector and presses the side flap in the direction of the channel axis 10. If the proposed folding area 4 is formed without the side flap 50 shown in FIGS. 3 to 9 or also without the step 48, this adaptation procedure is dispensed with.

Moreover, the contour of the used injection channel 2 does not necessarily have to be of a circular shape. Possible embodiments are shown in FIGS. 16 to 19 that show the possible geometrical shapes, but are not limited to these. For all geometrical shapes, it is crucially important that the distal end of the injection channel 2 has no sharp edges. Sharp edges on the outer surface pose an increased risk of injury to the eye of the patient, and sharp edges on the inside can cause damage of the IOL 56. The square contour shown in FIG. 18, with rounded edges, represents just one possible embodiment of these geometry variations and can in principle be extended to a basic shape with n corners, each with round edges.

When using an embodiment according to FIG. 1A or 1B, this is inserted with closed folding area 4, as shown for example in FIG. 9 or FIG. 15, into an open frontal area 67 of the injector housing 38, similar to that shown in FIG. 20. The open frontal area 67 is contiguous with a grip part 70. The initially wide opening merges into a longitudinal slit 69. The structural unit is connected in a sufficiently stable manner to the injector housing 38. For this purpose, provision is made, for example, for overdimension matching between the grip pieces 20 of the folding area 4 and the longitudinal slit 69. Thus, in the closed finished state, the folding area 4 is pushed, with the two grip pieces 20 folded together, into the longitudinal slit 69 in the direction of the channel axis 10. The half-shells 8a, 8b are guided and held in the frontal area 67. The injector housing 38 with the structural unit disposed therein, consisting of folding area 4 and injection channel 2, forms an injector. When using an injector 36 according to FIG. 2, an insertion procedure is omitted.

In the next stage, the push rod 44 is pushed forward in the direction of the channel axis 10 until the plunger 68, shown in FIG. 20 and disposed at the end of the push rod 44, makes contact with the folded IOL 56. This is then initially pushed out from the folding area 4 into the injection channel 2. The physician can then inject the IOL 56 into the prepared eye of the patient by first introducing the injection tip 32 of the injection channel 2 into an incision. By continuous pressure of the plunger 68 on the IOL 56, the latter can then be implanted into the prepared capsular bag of the eye of the patient. It does not matter whether this is done by direct pressure on the rear pressure plate 46 or by converting a radial turning movement into a linear movement (method steps not shown).

The appliances used for implantation are preferably made of a material suitable for disposable articles. Alternately, the injector housing 38 according to FIG. 20 can also be configured for repeated use. In this case, it must then be cleaned and sterilized after each use. In addition, the appliances used also have a suitable ergonomic configuration.

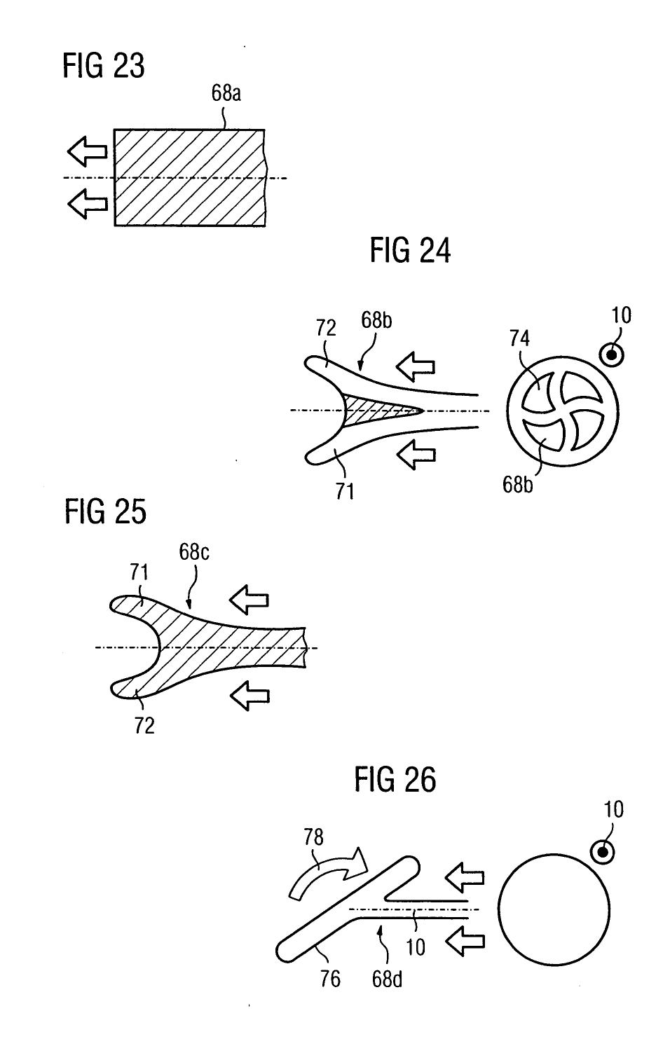

FIGS. 23 to 26 show various embodiments of the plunger 68. According to FIG. 23, a plunger 68a has a substantially cylindrical shape and is adapted to the guide channel 24 and to the injection channel 2. The two channels 2, 24 in this case preferably have the same internal diameter, and one which remains the same along the channel axis 10. The plunger 68a shown in FIG. 23 is specially configured for an only minimally conical injection channel 2. The three further embodiments allow the IOL 56 also to be pushed, with minimal risk of becoming stuck, through an injection channel 2 of greater conicity. The reason for this is that the respective plunger 68b-68d is deformable. According to FIGS. 24 and 25, the plunger 68b, 68c has a thickened head area 71 in whose central inner area a recess is formed so that an in particular annular plunger tip 72, which is easily compressible, protrudes at the edges. The embodiment shown in FIG. 24 differs from that in FIG. 25 in that cavities 74 in the inside of the plunger 68b permit a reduction in the radial cross section with minimal force. In the plunger 68d according to FIG. 26, the front end is provided with a pusher plate 76 that is set at an angle relative to the channel axis 10. The pusher plate 76 is therefore not perpendicular to the channel axis 10. If the injection channel contour narrows in the extent that a fairly circular cross-sectional profile changes to an increasingly elliptic cross-sectional profile toward the distal end, the pusher plate 76 can compensate for the proposed geometry by being pressed laterally in arrow direction 78, without leaving gaps with the injection channel 2.

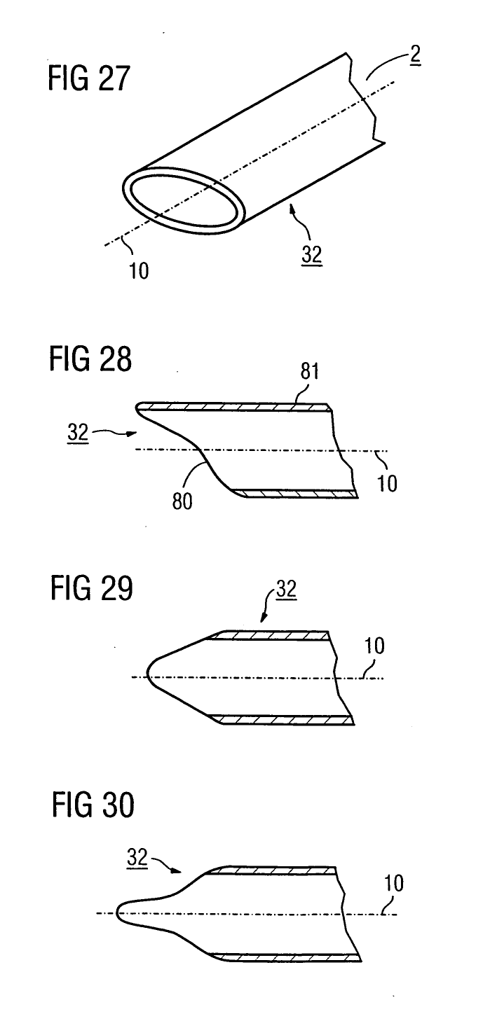

FIGS. 28 to 30 show various embodiment variants for the injection tip 32, these having a suitable shape for easy insertion into the eye. One possible embodiment is the beveling, shown in FIG. 1A, 1B and FIG. 2, of the distal end of the injection channel to form the injection tip 32, which is shown once again in FIG. 27. Further alternative configurations are shown in FIGS. 28 to 30.

The injection channel 2 according to FIG. 28 is not beveled in a straight line, as in FIG. 27, but instead on one side along a curving line 80. In a further variant of the tip of the injection channel 2, it is beveled on both sides toward the channel axis 10, as is shown in FIGS. 29 and 30. It is not necessary for the front most-tip to lie on the center axis of the channel. An asymmetrical tip is also possible. Whereas the tip is of rectilinear configuration in the embodiment in FIG. 29, it is curved in the manner shown in FIG. 30.

The embodiments shown permit two basic possibilities as regards insertion into the incision. On the one hand, when using geometries such as are shown in FIGS. 27 and 28, the operator can slightly lift the upper half of the incision in order to make room for the increasing cross section of the injection tip 32. Alternately, it is also possible for the incision to be drawn apart by the contour itself during axial advance of the injection tip 32 into the eye of the patient. The embodiments shown in FIGS. 29 and 30 are suitable for this purpose, and also the injection tip 32 according to FIG. 28 when turned through 180°, on insertion into the eye, so that its long side 81 is facing the patient's eye.

For producing the described injection system, it is recommended to use polymer materials together with the customary manufacturing methods. However, it is also generally possible to use other materials. Moreover, the material of the foldable IOL 56 to be implanted is not important to the functioning of the system, as long as the material satisfies the requirement of being foldable.

We claim:

1. A device for folding an intraocular lens, the device comprising:
a folding area having two pivotably interconnected half-shells which can be pivoted from an open starting position to a closed finishing position in which said half-shells enclose between them a guide channel for receiving the intraocular lens, said half-shells including a first half-shell and a second half-shell; and
a sheet-like band loop having a securing end secured on said first half-shell and guided displaceably by said second half-shell, and in the open starting position, a receiving space for the intraocular lens is formed between said band loop and said two half-shells such that, with the intraocular lens fitted in place, the intraocular lens is surrounded by said band loop and said two half-shells in the open starting position, and in that, by pulling on said band loop, said two half-shells being moved in a direction toward the closed finishing position such that the intraocular lens is folded.

2. The device according to claim 1, wherein said two half-shells have outer longitudinal edges, and in the closed finishing position said two half-shells bear against one another along said outer longitudinal edges, said band loop is secured on said longitudinal edge of said first half-shell and is guided on said longitudinal edge of said second half-shell.

3. The device according to claim 1, wherein said second half-shell has a guide slit formed therein for guiding said band loop.

4. The device according to claim 1, wherein in that, viewed in a cross section perpendicular to a channel axis of said two half-shells, an inner side of one of said two half-shells has a step for supporting an edge of the intraocular lens.

5. The device according to claim 4, wherein one of said two half-shells has a side flap extending along the channel axis and can be guided, from a forward position in which said step is formed, radially toward the channel axis into a retracted position.

6. The device according to claim 5, wherein said side flap can be locked in the retracted position.

7. The device according to claim 1, wherein said band loop has a remaining portion and a subsidiary portion attached to said securing end and, at a kink oriented parallel to said guide channel, said subsidiary portion is connected in an articulated manner to said remaining portion of said band loop, and said remaining portion, in the open starting position, is oriented outward in a radial direction so that said receiving space has a pocket extending above said first half-shell.

8. The device according to claim 1, further comprising a gripping device selected from the group consisting of a grip piece and a grip stub, said gripping device adjoining at least one of said two half-shells.

9. The device according to claim 1, wherein said band loop is configured to be secured on an outer side of said second half-shell.

10. The device according to claim 8, wherein said band loop is configured to be secured on an outer side of said gripping device.

11. The device according to claim 1, wherein said band loop is guided on said second half-shell in such a way as to exclude a possibility of said band loop sliding back counter to a pull direction of said band loop.

12. The device according to claim 1, wherein at least one of said two half-shells and said band loop has at least one through-bore formed therein for introducing a viscoelastic material.

13. The device according to claim 1, further comprising an injection channel, said injection channel and said folding area forming a one-piece structural unit and said injection channel is flush with said guide channel.

14. The device according to claim 13, further comprising an injector housing having a push rod and a push channel for receiving said push rod, said folding area, said injection channel and said injector housing forming a one-piece structural unit in which said folding area is disposed between said push channel and said injection channel.

15. The device according to claim 1, further comprising a deformable plunger for pushing the intraocular lens forward in said guide channel.

16. The device according to claim 13, wherein said injection channel has a front end with an injection tip narrowed substantially to a channel center of said injection channel.

17. The device according to claim 1, further comprising a container, and in the open starting position with the intraocular lens fitted into said receiving space, said folding area containing the intraocular lens is disposed for storage and/or transport in said container.

18. The device according to claim 17, wherein said container is filled with a liquid and is sealed off tight.

19. The device according to claim 17, wherein the intraocular lens is stored dry in said container, and said container is sealed off tight.
